(19) United States
(12) Patent Application Publication
(10) Pub. No.: US 2021/0405025 A1
(43) Pub. Date: Dec. 30, 2021

(54) IDENTIFYING COMPOUNDS FOR TREATING CANCER AND USE THEREOF

(71) Applicant: THE UAB RESEARCH FOUNDATION, Birmingham, AL (US)

(72) Inventor: Christopher Klug, Birmingham, AL (US)

(73) Assignee: THE UAB RESEARCH FOUNDATION, Birmingham, AL (US)

(21) Appl. No.: 17/251,519

(22) PCT Filed: Jun. 14, 2019
(86) PCT No.: PCT/US2019/037195

§ 371 Date: Dec. 11, 2020

Related U.S. Application Data
(60) Provisional application No. 62/684,921, filed on Jun. 14, 2018.

(51) Int. Cl. G01N 33/50

(52) U.S. Cl. CPC G01N 33/5011

(57) ABSTRACT

Provided herein is a method for identifying compounds that inhibit cell growth or have cytotoxic activity against cancer cells, such as hematological cancer cells or ovarian cancer cells, in a subject. Further provided is a method for identifying one or more compounds that sensitize refractory cancer cells from a subject. Also provided are compositions and methods for treating cancer, including refractory cancer, such as refractory acute myeloid leukemia (AML) or ovarian cancer.

### BACKGROUND

In the United States alone, over 1.7 million new cases of cancer are diagnosed each year and over 500,000 people die each year from cancer. There are numerous types and subtypes of cancers that account for these statistics. By way of example, acute myeloid leukemia (AML) is the most common acute leukemia in humans, accounting for an estimated 10,400 deaths annually due to development of chemorefractory disease. AML has a five-year survival rate of 26 percent. Therefore, compositions and methods for treating AML and other cancer types are needed. Also needed are methods of identifying agents that are useful in the treatment of various forms of cancer, including hematologic cancers (e.g., leukemia) and ovarian cancer.

### SUMMARY

Provided herein is a method for identifying compounds that inhibit cell growth or have cytotoxic activity against AML or other leukemic or cancer cells in a subject. The method comprises obtaining bone marrow or peripheral blood cells from a subject having AML or other hematological cancer; pre-culturing the bone marrow or peripheral blood cells for a short period (e.g., at least 12 hours) that allows cell death of a subset of the cells that do not adapt to the culture conditions or fail to survive in culture; sorting the pre-cultured cells through a cell sorter to acquire a subset of viable cancer cells (e.g., a relatively pure population of viable leukemic cells); dispensing aliquots of the viable cells into individual wells, for example, two or more wells, of one or more multi-well tissue culture plates; culturing the cells in each well; contacting the cells in each well with a unique compound selected from the compounds set forth in Table 1 or Table 2; identifying one or more compounds that inhibit cell growth or promote cell death of the contacted cells.

Also provided herein is a method for identifying one or more compounds that sensitize refractory cancer cells (e.g., hematological cancer cells or ovarian cancer cells) from a subject to a low dose of the therapeutic agent to which the cells are refractory. The low dose is optionally the IC₁₀ concentration of the therapeutic agent in inhibiting cell growth or promoting cytotoxicity of the hematological cancer cells from bone marrow or peripheral blood cells from the subject having a hematological cancer or ovarian cancer cells from the subject with ovarian cancer. Hematological cancer cells from bone marrow or peripheral blood cells from a subject having a hematological cancer or ovarian cancer cells from a subject with ovarian cancer are obtained, wherein the hematological or ovarian cancer cells are refractory to a therapeutic agent. Aliquots of the cells (optionally after freezing, thawing, pre-culturing, and sorting for selection of a relatively pure population of viable cells) are dispensed into individual wells of one or more multi-well tissue culture plates and the cells in a first subset of the wells is contacted with a combination of a low dose of the therapeutic agent to which the cells are refractory and a selected dose of a unique compound selected from the compounds set forth in Table 1 or Table 2, wherein the selected dose of each compound varies across wells in the first subset. The cells in a second subset of the wells is contacted with the same compounds and selected doses thereof of the first subset (without the therapeutic agent). One or more compounds that sensitize the cells to the refractory agent are identified by determining which compounds inhibit cell growth or promote cytotoxicity in the first subset of well of the contacted cells more than the same one or more compounds in the second subset of wells. For example, a dose-response curve can be derived to determine whether the IC₅₀ for the compound has shifted (i.e., the IC₅₀ is lower) in the presence of the low dose of the therapeutic agent as compared to the absence of the therapeutic agent.

The method for identifying one or more compounds that sensitize refractory cancer cells (e.g., hematological cancer cells or ovarian cancer cells) from a subject optionally includes the steps for determining the selected low dose of the therapeutic agent by dispensing aliquots of the cells (optionally after freezing, thawing, pre-culturing, and sorting for selection of a relatively pure population of viable cells) into individual wells of one or more multi-well tissue culture plates; contacting sets of wells with multiple concentrations of the therapeutic agent (e.g., at least a first concentration, a second concentration, a third concentration, or a fourth concentration, and up to at least ten concentrations) of the therapeutic agent; determining which concentrations of the therapeutic agent inhibit cell growth or promote cytotoxicity to determine the IC₅-IC₂₅ (e.g., IC₁₀) of the therapeutic agent.

The methods as disclosed herein include the important step of acquiring a relatively pure population of viable hematological cancer cells or ovarian cancer cells through the pre-culturing and sorting steps. These steps ensure a reduction in background noise, including background noise associated with non-viable cancer cells, background noise associated with non-cancer cells (which can be a significant portion of the biological samples), and background noise associated with death of cells that do not adapt to culture. Reduction in background results in a highly sensitive assay system, allowing the identification of sensitivities to specific single compounds or combinations of drugs that would otherwise not be possible.

Methods of treating hematological cancer, such as leukemia (e.g., AML), or ovarian cancer in a subject are provided that comprise administering to the subject an effective amount of one or more compounds set forth in Table 1 or Table 2, optionally in combination with one or more anti-cancer therapies. In some methods, the subject (or cancer cells) are refractory to one or more chemotherapeutic agents or molecularly targeted agents. Optionally, one or more anti-cancer therapies is administered in combination with the agent to which the cancer cells are refractory.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows the results of a representative screening assay in a 1,536-well plate where AML cell viability was assessed after 3 days of culturing cells in independent wells, each well containing an independent, approved drug used at 10 μM concentration. Approximately 2,174 compounds were evaluated in this screen, with drugs purchased from the chemical compounding companies Selleck (Houston, Tex.), Microsource (Gaylordsville, Conn.) and Enzo (Farmingdale, N.Y.) in order to ensure comprehensive coverage of the approved compound library. Color-scale indicates relative cell viability in each well (red >90% alive, blue >90% dead), shown in gray scale on the plate.

FIG. 2 shows identification of all approved drugs with ability to kill >70% of relapsed AML cells in a 3-day culture. After evaluation of 18 total AML cases, saturation for identification of all approved drugs with activity against relapsed AML (412 total compounds) was approached.

FIG. 3 is a bar graph summarizing the number of drugs that were active against the total number of AML cases. Fifty-two drugs were active against all 18 cases, while 115 total drugs were only active in 1 out of 18 patient samples. The number of drugs active against varying numbers of AML cases is shown. These results show that the majority of identified drugs have patient-specific cytotoxic activity.

FIG. 4 illustrates a rapid method for identification of drug combinations that re-sensitize FLT3-ITD-mutated AML patient cells to killing using targeted FLT3 inhibitors as an example. This example of the method evaluates a total of 256 drugs taken from the list in Table 1. It shows that, when a very low dose (the IC₁₀) of the drug to which the patient has become resistant in the clinic is added to one set of duplicate plates containing all 256 drugs arrayed in dose-response, only two drugs caused the cells to be more sensitive to killing in the 3-day co-culture of cells with drugs. This is evidenced by a lower IC₅₀ value when the combination is compared to the IC₅₀ value when either drug is used alone. Importantly, these two drugs target the same molecule, JAK2, which was subsequently determined to be mutated (JAK2^{V617F}) when the patient samples were DNA sequenced. This shows that mutation of JAK2 is associated with drug resistance to the targeted FLT3-ITD inhibitor in this patient.

### DETAILED DESCRIPTION

Methods for Identifying Compounds with Growth Inhibitory Activity and/or Cytotoxic Activity Against Leukemic Cells from a Specific Subject

Provided herein are highly reproducible assays for identifying compounds with growth inhibitory activity and/or cytotoxic activity against hematological cancer cells, such as leukemic cells, from a specific subject (e.g., a subject with AML). By way of example, the method comprises first obtaining a biological sample (e.g., bone marrow or peripheral blood cells) containing cancer cells from a subject having a hematological cancer (e.g., AML or other leukemia). The next step is a pre-culturing step in which the cells from the biological sample are pre-cultured (e.g., in a plastic flask) for a period of time that allows cell death of a subset of the cells (e.g., cells that do not adapt to or survive the culture conditions). After the pre-culturing step, the pre-cultured cells are sorted through a cell sorter to acquire a subset of viable cancer cells (e.g., leukemic cells). The sorting step results in a relatively pure population of viable cancer cells that are dispensed in aliquots into individual wells of one or more multi-well tissue culture plates and the cells are cultured. The cells in each culture well are then contacted with a unique compound or unique set of compounds selected from the compounds set forth in Table 1 or Table 2 to identify the compounds or sets of compounds that inhibit cell growth or promote cell death of the contacted cells. It is only through this combination of steps, including the critical steps of pre-culturing and sorting, that the background noise is sufficiently low to allow the sensitivity and accuracy needed to screen a subject's cells so as to identify which compound, alone or in combination, are useful in treating the subject's leukemia. Thus, numerous compounds can be screened using cells from a single subject and the effective compound or set of compounds can be selected that is effective for that subject's cancer cells. The method thus provides the opportunity for enhancing the selection of treatment for a specific subject and for resulting in a higher likelihood of clinical success.

TABLE 1

| | #Cases >70% | |
|---|---|---|
| Drug name | cell death | Broad Class/protein target |
| (−)-BUTACLAMOL HCL | 3 | Dopamine D1 receptor inhibitor |
| 10-DAB | 1 | Chemotherapeutic, precursor for docetaxel |
| 10- | 12 | Chemotherapeutic |
| HYDROXYCAMPTOTHECIN | | |
| 17- | 2 | Cortisol precursor |
| HYDROXYPROGESTERONE | | |
| 2-METHOXYESTRADIOL | 2 | Angiogenesis inhibitor, natural metabolite of estradiol |
| 4-AMINOPYRIDINE | 1 | Treatment of multiple sclerosis |
| 8-AZAGUANINE | 1 | Purine analog, chemotherapeutic |
| A-85783 | 9 | Platelet activating factor receptor inhibitor |
| ABACAVIR SULFATE | 1 | HIV reverse transcriptase inhibitor |
| ABAMECTIN | 18 | Macrocyclic lactone |
| ABT-239 | 1 | H3-receptor inverse-agonist, neurologic diseases |
| ABT-751 | 5 | Microtubules, antimitotic sulfonamide that binds to the colchicine- |
| | | binding site on beta-tubulin and inhibits the polymerization of |
| | | microtubules |
| ACENOCOUMAROL | 4 | Vitamin K antagonist (anticoagulant, warfarin) |
| ACEPROMAZINE MALEATE | 2 | Phenothiazine derivative antipsychotic drug |
| ACIPIMOX | 1 | Niacin receptor 1, inhibits triglyceride lipase |
| ACRIFLAVINIUM | 5 | Antiseptic; antiparasitic; HIF-1 inhibitor |
| HYDROCHLORIDE | | |
| ACRISORCIN | 18 | Antifungal |
| ADAROTENE | 11 | Atypical retinoid |
| ADEFOVIR | 18 | Reverse Transcriptase (HepB) |
| AEE-788 | 13 | HER1/2, EGFR1/2, KDR inhibitor |
| ALBENDAZOLE | 9 | Antiparasitic, microtubules |
| ALBUTEROL | 1 | Beta2 adrenergic receptor agonist |
| ALEXIDINE | 18 | Antimicrobial |
| ALVERINE CITRATE | 4 | Smooth muscle relaxant |
| AMCINONIDE | 1 | Topical glucocorticoid |
| AMG-073 HCL | 1 | Calcimimetic/reduces parathyroid hormone synthesis |
| AMINACRINE | 18 | Antiseptic (topical) |
| AMLODIPINE | 14 | Calcium ion channel blocker/lowers blood pressure |
| AMONAFIDE MALATE | 8 | Chemotherapeutic, topo II inhibitor |
| AMOROLFINE | 9 | Antifungal, morpholine antifungal drug that inhibits Δ¹⁴-sterol |
| | | reductase |
| AMSACRINE | 4 | Chemotherapeutic |
| ANCITABINE | 14 | Chemotherapeutic |
| HYDROCHLORIDE | | |
| ANHYDROVINBLASTINE | 5 | Microtubule inhibitor |
| ANTIMONY POTASSIUM | 3 | Antiparasitic, emetic |
| TARTRATE TRIHYDRATE | | |
| APILIMOD MESYLATE | 4 | IL-12/23 inhibitor |
| APO-866 | 17 | NAMPT (nicotinamide phosphoribosyltransferase |
| | | inhibitor)(NAD biosynthesis inhibitor) |
| APOMORPHINE | 5 | Dopamine agonist |
| ARSENIC TRIOXIDE | 16 | Differentiation inducer |
| DIETHANOLAMINE SALT | | |
| ARTESUNATE | 5 | Antiparasitic/antimalarial |
| ASTEMIZOLE | 7 | Histamine H1 receptor antagonist |
| AT-7519 | 18 | CDK 1, 2, 4, 6, 9 inhibitor |
| ATENOLOL | 1 | Beta adrenergic blocker |
| ATORVASTATIN CALCIUM | 12 | Statin |
| AURANOFIN | 17 | Antiparasitic, rheumatoid arthritis |
| AVN944 | 10 | IMPDH (Inosine 5-monophosphate dehydrogenase II) inhibitor |
| AXITINIB | 9 | VEGFR, cKIT, PDGFR inhibitor |
| AZACYTIDINE | 8 | Chemotherapeutic |
| AZAPERONE | 1 | Sedative, anti-psychotic |
| AZATHIOPRINE | 10 | Immunosuppressant |
| AZD-1152 | 5 | Aurora kinase B inhibitor |
| AZD-1480 | 12 | JAK2, JAK3, TYK2 inhibitor |
| AZD-5438 | 17 | CDK 1, 2, 9 inhibitor |
| AZD-6482 | 1 | PI3K beta inhibitor |
| AZD-7762 | 14 | Chk1/2 inhibitor |
| AZD8330 | 2 | MEK 1/2 inhibitor |
| AZELASTINE | 2 | H1-receptor/anti-histamine |
| AZELNIDIPINE | 5 | Calcium ion channel blocker |
| AZITHROMYCIN | 1 | Antibiotic, antiseptic |
| BARDOXOLONE | 12 | NF-kB inhibitor, activator of KEAP1-Nrf2 pathway |
| BAZEDOXIFENE HCL | 8 | Estrogen receptor modulator |
| BECATECARIN | 18 | Chemotherapeutic |
| BECLOMETHASONE | 1 | Corticosteroid, anti-inflammatory |
| DIPROPIONATE | | |
| BELINOSTAT | 1 | HDAC inhibitor |
| BENOXINATE | 5 | Anesthetic |
| BENZALKONIUM CHLORIDE | 15 | Cationic surfactant |
| BENZETHONIUM CHLORIDE | 16 | Antiseptic |
| BENZYDAMINE | 4 | NSAID/anti-inflammatory |
| BERBERINE CHLORIDE | 1 | Isoquinoline alkaloid and active component of various Chinese |
| | | herbs, with potential antineoplastic, radiosensitizing, anti- |
| | | inflammatory, anti-lipidemic and antidiabetic activities |
| BETAMETHASONE | 1 | Steriod anti-inflammatory |
| BIFONAZOLE | 1 | Antifungal |
| BINOSPIRONE MESYLATE | 1 | Serotonin 5-HT1A receptor agonist |
| BISOCTRIZOLE | 1 | Sunscreen |
| BLEOMYCIN | 2 | Chemotherapeutic |
| BMS-387032 | 18 | CDK 2, 7, 9 inhibitor |
| BMS-754807 | 1 | IGF-1R receptor inhibitor |
| BORTEZOMIB | 18 | Proteosome inhibitor |
| BOSUTINIB | 12 | BCR-ABL, SRC |
| BROMHEXINE | 1 | Mucolytic |
| BROXALDINE | 2 | Antiprotozoal |
| BUDESONIDE | 2 | Corticosteroid |
| BUFEXAMAC | 9 | HDAC 6 and 10 inhibitor, anti-inflammatory |
| BUTACAINE | 1 | Chemotherapeutic, anesthetic |
| BUTOCONAZOLE | 1 | Antifungal |
| CABAZITAXEL | 5 | Chemotherapeutic |
| CABOZANTINIB | 2 | MET, VEGFR2, AXL, and RET inhibitor |
| CAMPTOTHECIN | 12 | Chemotherapeutic |
| CAPSAICIN | 1 | Binds to vanilloid receptor subtype 1 |
| CARFILZOMIB | 18 | Proteosome inhibitor |
| CEPHALOMANNINE | 7 | Chemotherapeutic |
| CERIVASTATIN | 17 | Statin |
| CETRIMONIUM BROMIDE | 17 | Topical antibacterial, antifungal |
| CETYLPYRIDINIUM | 18 | Antibiotic, antiseptic |
| CHLORIDE | | |
| CH5132799 | 3 | PI3Kalpha inhibitor |
| CHLORHEXIDINE | 11 | Antiseptic, MMP2 and 9 inhibitor |
| DIHYDROCHLORIDE | | |
| CHLORMIDAZOLE | 1 | Antifungal |
| CHLOROXINE | 1 | Antibacterial |
| CICLETANINE | 4 | Diuretic used for hypertension |
| CICLOPIROX | 14 | Antifungal |
| CILNIDIPINE | 1 | L-type and N-type calcium channel blocking functions. |
| CINACIGUAT | 1 | Activates soluble guanylate cyclase, which is nitric oxide receptor |
| CLADRIBINE | 17 | Chemotherapeutic, Purine analog |
| CLEMASTINE | 1 | Antihistamine, H1-receptor antagonist |
| CLEMIZOLE | 1 | Histamine H1 receptor antagonist |
| HYDROCHLORIDE | | |
| CLIMBAZOLE | 1 | Antifungal |
| CLIOQUINOL | 6 | Antiparasitic, antifungal, neuotoxic at high doses |
| CLOBETASOL PROPIONATE | 4 | Corticosteroid |
| CLOFARABINE | 18 | Chemotherapeutic, Purine analog |
| CLOFAZIMINE | 6 | Antibacterial |
| CLOMIFENE | 10 | Anti-oestrogen, ovulatory stimulant |
| CNF-2024 | 18 | HSP90 inhibitor |
| COLCHICINE | 13 | Microtubule inhibitor |
| CP-945 | 1 | Cannabinoid type 1 (CB1) receptor inhibitor |
| CRIZOTINIB | 10 | ALK and ROS1 inhibitor |
| CURCUMIN | 1 | HDAC 1,3,8 inhibitor |
| CYCLOCYTIDINE | 13 | Chemotherapeutic, pyrimidine analog |
| CYCLOHEXIMIDE | 18 | Protein synthesis |
| CYCLOSPORINE | 10 | Calcineurin and P-glycoprotein inhibitor |
| CYPROTERONE | 1 | Antiandrogen, progestogen |
| CYT387 | 14 | JAK1/2 inhibitor |
| CYTARABINE | 16 | Chemotherapeutic |
| D-CYCLOSERINE | 1 | Agonist of glycine recognition component of glutamatergic NMDA |
| | | receptor. |
| DABRAFENIB | 4 | BRAF inhibitor |
| DACARBAZINE | 14 | Chemotherapeutic |
| DACTINOMYCIN | 17 | Chemotherapeutic |
| DANAZOL | 1 | Steriod for treatment of endometriosis |
| DARAPLADIB | 18 | Phospholipase A2 inhibitor |
| DASATINIB | 6 | BCR-ABL, SRC, Ephrin inhibitor |
| DAUNORUBICIN | 18 | Chemotherapeutic |
| DEFLAZACORT | 8 | Glucocorticoid/anti-inflammatory |
| DEQUALINIUM CHLORIDE | 17 | PKC inhibitor and K-channel blocker |
| DESLORATADINE | 1 | H1-receptor antagonist, antihistamine |
| DEXAMETHASONE | 2 | Corticosteroid |
| DEXNIGULDIPINE | 8 | Calcium channel blocker and a₁-adrenergic receptor antagonist |
| DIFLUPREDNATE | 1 | Corticosteroid |
| DIGITOXIN | 18 | Cardiac Glycoside |
| DIGOXIGENIN | 18 | Cardiac Glycoside |
| DIGOXIN | 18 | Cardiac Glycoside |
| DIHYDROERGOTAMINE | 1 | Seratonin receptor agonist, also adrenergic and dopamine |
| MESYLATE | | |
| DILAZEP | 2 | Vasodilator, antiplatelet, adenosine uptake inhibitor |
| DIHYDROCHLORIDE | | |
| DIMESNA | 1 | Uroprotective |
| DIPYRIDAMOLE | 2 | Phosphodiesterase-4 inhibitor |
| DOCETAXEL | 5 | Chemotherapeutic |
| DOMIPHEN BROMIDE | 16 | Antiseptic |
| DONEPEZIL (Aricept) | 3 | AChE inhibitor |
| DORAMECTIN | 18 | Macrocyclic lactone |
| DORZOLAMIDE | 4 | Carbonic anyhdrase inhibitor |
| DOXOFYLLINE | 1 | Xanthine bronchodilator |
| DOXORUBICIN | 18 | Chemotherapeutic |
| DRONEDARONE | 12 | Antiarrhythmic |
| DROPERIDOL | 2 | Antidopaminergic drug |
| DYDROGESTERONE | 1 | Synthetic progesterone |
| ELACRIDAR | 2 | MDR-1/BCRP inhibitor, dual inhibitor of P-glycoprotein (MRP-1, |
| | | ABCB1) and breast cancer resistance protein (BCRP, ABCG2) |
| ELIPRODIL | 1 | NMDA antagonist |
| EMETINE | 18 | Antiprotozoal, blocks protein synthesis |
| EPIRUBICIN | 18 | Chemotherapeutic |
| EPRINOMECTIN | 5 | Macrocyclic lactone |
| ERLOTINIB | 6 | EGFR inhibitor |
| ESTRADIOL CYPIONATE | 2 | Estrogen steroid, hormone replacement |
| ESTROPIPATE | 1 | Estrogen used as hormone replacement in menopausal women |
| ETHACRIDINE LACTATE | 2 | Antiseptic |
| ETHINYL ESTRADIOL | 1 | Synthetic estrogen |
| ETHOXZOLAMIDE | 2 | Carbonic anhydrase inhibitor/anti-TB agent |
| ETICLOPRIDE | 1 | Dopamine D2/D3 receptor antagonist |
| HYDROCHLORIDE | | |
| ETIFOXINE | 4 | Non-benzodiazepine anxiolytic |
| ETOPOSIDE | 5 | Chemotherapeutic |
| ETRAVIRINE | 1 | Reverse transcriptase inhibitor |
| ETRETINATE | 1 | Retinoid |
| EXATECAN MESYLATE | 12 | Chemotherapeutic |
| FENBENDAZOLE | 5 | Antiparasitic |
| FENDILINE | 1 | inhibits KRAS membrane localization; Adrenergic antagonist, |
| HYDROCHLORIDE | | calcium channel blocker |
| FENOFIBRATE | 3 | PPARα agonist |
| FENRETINIDE | 9 | Retinoid derivative |
| FENSPIRIDE | 3 | antitussive |
| FLECAINIDEACETATE | 1 | Antiarrhythmic |
| FLOXURIDINE | 8 | Chemotherapeutic |
| FLUBENDAZOLE | 2 | Antihelmintic |
| FLUCONAZOLE | 1 | Antifungal |
| FLUDARABINE | 16 | Chemotherapeutic |
| FLUDARABINE PHOSPHATE | 4 | Chemotherapeutic |
| FLUMETHASONE | 2 | Corticosteroid |
| FLUNARIZINE | 3 | Calcium ion channel inhibitor/migraine |
| FLUOCINONIDE | 1 | Corticosteroid cream |
| FLUOROMETHOLONE | 2 | Corticosteroid |
| FLUOROURACIL | 4 | Chemotherapeutic |
| FLUPHENAZINE | 3 | Dopamine D2 receptor antagonist |
| FLURANDRENOLIDE | 18 | Steroid |
| FLUTAMIDE | 2 | Nonsteroidal antiandrogen |
| FLUTICASONE PROPIONATE | 3 | Corticosteroid |
| FLUVASTATIN | 7 | Statin |
| FLUVOXAMINE MALEATE | 1 | Serotonin (5-HT) reuptake inhibitor |
| FLUXININ | 3 | Anti-inflammatory (NSAID) |
| FOROPAFANT | 6 | PAF (platelet-activating factor) antagonist |
| GEFITINIB | 2 | EGFR inhibitor |
| GEMCITABINE | 16 | Chemotherapeutic |
| GENTIAN VIOLET | 18 | Dye, antifungal, antibacterial |
| GFKI-258 | 16 | VEGR, FGF, PDGFR inhibitor |
| GLICLAZIDE | 1 | Increases insulin release/antidiabetic |
| GMX1778 | 16 | NAMPT (NAD biosynthesis inhibitor) |
| GRAMICIDIN (GRAMICIDIN | 9 | Antibiotic |
| A SHOWN) | | |
| GSK 461364 | 9 | Plk1 (polo-like kinase) inhibitor |
| GSK1070916 | 11 | Aurora B/C kinase inhibitor |
| GSK923295 | 8 | CENP-E kinesin motor ATPase inhibitor |
| GUANFACINE HCL | 1 | Selective alpha A2 receptor agonist |
| HALOBETASOL | 3 | Corticosteroid |
| PROPIONATE | | |
| HARMINE | 3 | MAO-A inhibitor, alkaloid |
| HKI 357 | 4 | EGFR and HER2 inhibitor |
| HMN-214 | 5 | PLK-1 (polo-like kinase) inhibitor |
| HOMIDIUM BROMIDE | 1 | Antiparasitic |
| HSP-990 | 17 | HSP90 inhibitor |
| HYCANTHONE | 9 | Antiparasitic |
| HYDROXYPROGESTERONE | 2 | Pregnane steroid, progesterone derivative |
| IBANDRONATE | 1 | Potent bisphosphonate, inhibits osteoclast bone resorption |
| IBRUTINIB | 2 | BTK, VEGFR, EGFR, RET |
| IDARUBICIN | 18 | Chemotherapeutic |
| IFENPRODIL TARTRATE | 4 | NMDA receptor antagonist |
| IODOQUINOL | 18 | Antiparasitic |
| ISONIAZID | 1 | Antibiotic for TB |
| ISPINESIB MESYLATE | 2 | Specific and reversible inhibitor of kinesin spindle protein (KSP) |
| ITRACONAZOLE | 6 | Antifungal |
| IVERMECTIN | 18 | Macrocyclic lactone |
| JNJ-28312141 | 7 | CSF1R (M-CSFR) inhibitor |
| JTC-801 | 18 | Nociceptin receptor antagonist |
| K201 | 4 | Antiarrythmic; calcium regulator |
| KETOCONAZOLE | 5 | Antifungal |
| KW-2449 | 9 | FLT3, BCR-ABL inhibitor (multikinase) |
| LACIDIPINE | 2 | Calcium channel blocker |
| LANATOSIDE C | 18 | Cardiac Glycoside |
| LAPATINIB | 2 | Dual EGFR and HER2/NEU inhibitor |
| LAQ-824 | 18 | HDAC inhibitor |
| LASALOCID SODIUM | 10 | Antibiotic |
| LATANOPROST | 1 | Treatment of high blood pressure in the eye |
| LEXIBULIN | 13 | Microtubules, microtubule polymerization inhibitor |
| LOMERIZINE | 7 | Calcium ion channel blocker |
| LOVASTATIN | 2 | Statin |
| LY333531 | 12 | PKC beta 1/2 inhibitor |
| MANIDIPINE | 5 | Calcium ion channel blocker |
| MEBENDAZOLE | 5 | Antiparasitic |
| MEBHYDROLIN | 1 | H1-receptor antagonist/anti-histamine |
| MEGLUMINE | 1 | Glucose derivative |
| MELENGESTROL ACETATE | 2 | Steroidal progestin |
| MELPHALAN | 4 | Chemotherapeutic/alkylating agent |
| MERCAPTOPURINE | 10 | Chemotherapeutic |
| METHYLBENZETHONIUM | 16 | Anti-infective |
| CHLORIDE | | |
| METHYLENE BLUE | 18 | Guanylate cyclase inhibitor |
| METHYLPREDNISOLONE | 1 | Glucocorticoid |
| MEVASTATIN | 7 | Statin |
| MICONAZOLE | 1 | Antifungal (topical) |
| MITOXANTRONE | 18 | Type II topoisomerase inhibitor |
| MK-2461 | 2 | MET inhibitor |
| MLN9708 | 1 | Proteosome inhibitor |
| MOMETASONE FUROATE | 2 | Corticosteroid |
| MONENSIN SODIUM | 7 | Antiparasitic/antibiotic, cation exchange across membranes |
| (MONENSIN A IS SHOWN) | | |
| MUBRITINIB | 1 | HER2 inhibitor |
| MYCOPHENOLATE | 5 | Immunosuppressant |
| MOFETIL | | |
| MYCOPHENOLIC ACID | 5 | Immunosuppressant/inosine monophosphate inhibitor |
| NALTRIBEN | 1 | Selective δ2 opioid receptor (DOR) antagonist |
| METHANESULFONATE | | |
| HYDRATE | | |
| NARASIN | 8 | Antiparasitic, antibacterial derivative of salinomycin |
| NEFAZODONE | 1 | Antidepressant |
| NEFOPAM | 2 | Analgesic |
| NICLOSAMIDE | 1 | STAT inhibitor, antiparasitic |
| NIGULDIPINE | 12 | Calcium channel blocker, a1-adrenergic receptor antagonist |
| NOLATREXED | 1 | Chemotherapeutic, quinazoline folate analog |
| DIHYDROCHLORIDE | | |
| NONOXYNOL-9 | 2 | Surfactant |
| NVP-BGT226 | 8 | Class I PI3K/mTOR inhibitor for PI3Kα/β/γ |
| OBATOCLAX | 2 | Bcl2 inhibitor |
| OCTOCRYLENE | 1 | Sunscreen, cosmetic |
| ONDANSETRON (Zofran) | 1 | 5-HT3 receptor antagonist, antinausea |
| ORLISTAT | 9 | Lipase inhibitor |
| OSI-420 | 3 | EGFR inhibitor |
| OSIMERTINIB (tagrisso) | 1 | EGFR inhibitor |
| OTILONIUM BROMIDE | 4 | AChR, antimuscarinic and calcium channel blocker |
| OUABAIN | 18 | Cardiac Glycoside |
| OXELAIDIN CITRATE | 2 | Antitussive |
| OXETHAZAINE | 1 | Local anesthetic |
| OXIBENDAZOLE | 5 | Antiparasitic |
| OXICONAZOLE | 1 | Antifungal |
| PACLITAXEL | 5 | Chemotherapeutic, microtubules |
| PADEXOL | 5 | Chemotherapeutic |
| PANOBINOSTAT | 1 | Non-selective histone deacetylase inhibitor |
| PAZOPANIB | 4 | RTK inhibitor/c-KIT, FGFR, PDGFR, VEGFR |
| PAZUFLOXACIN | 1 | Antibacterial, fluoroquinolone |
| PENFLURIDOL | 14 | Dopamine receptor inhibitor used in schizophrenia |
| PENTAMIDINE | 2 | Antimicrobial, antiparasitic, antifungal |
| PERHEXILINE MALEATE | 13 | CPT1 and CPT2 inhibitor, anti-anginal agent |
| PERPHENAZINE | 2 | Antipsychotic |
| PF-00477736 | 13 | CHK1 inhibitor |
| PF-04928473 | 18 | HSP90 inhibitor |
| PF-4457845 | 1 | Fatty acid amide hydrolase inhibitor |
| PF-562271 | 18 | FAK inhibitor |
| PHA-690509 | 11 | CDK 2, 4 inhibitor |
| PHA-767491 | 18 | CDK 7, 9 inhibitor |
| PHA-793887 | 16 | CDK 2, 5, 7 inhibitor |
| PHENYLMERCURIC | 18 | Antifungal |
| ACETATE | | |
| PHTHALYLSULFATHIAZOLE | 1 | Antimicrobial |
| PIMECROLIMUS | 2 | Calcineurin inhibitor |
| PIMOZIDE | 1 | Antipsychotic |
| PIOGLITAZONE | 8 | Chemotherapeutic |
| PIROCTONE OLAMINE | 13 | Antifungal, shampoo |
| PIROMIDIC ACID | 5 | Antibiotic: quinolone |
| PITAVASTATIN CALCIUM | 13 | Statin |
| PLINABULIN | 12 | Microtubule inhibitor, JAK activation |
| PODOFILOX | 13 | Microtubule destabilizer |
| PONATINIB | 18 | BCR-ABL inhibitor |
| POSACONAZOLE | 6 | Antifungal |
| PRALATREXATE(FOLOTYN | 5 | DHFR inhibitor: Antifolate |
| PRAMOXINE | 1 | Anesthetic, antipuritic, cream |
| PRAVASTATIN | 1 | Statin |
| PREDNISOLONE | 1 | Steroid |
| PRIMAQUINE | 12 | Antiparasitic |
| PROBUCOL | 4 | Lowers HDL and LDL |
| PROCHLORPERAZINE | 6 | Seratonin receptor agonist, dopamine receptor antagonist |
| PROFLAVINE | 3 | Antibacterial |
| HEMISULFATE | | |
| PROPAFENONE | 1 | Antiarrhythmic agent |
| PROPARACAINE | 1 | Anesthetic |
| PROPOXYCAINE | 1 | Anesthetic |
| HYDROCHLORIDE | | |
| PROSCILLARIDIN | 18 | Cardiac Glycoside |
| PRX-8066 | 1 | Serotonin 2B receptor (5-HT2B) inhibitor |
| PUROMYCIN | 18 | Antibiotic |
| PYRITHIONE ZINC | 7 | Antifungal/Antibacterial |
| PYRONARIDINE | 9 | Antiparasitic, antimalarial |
| TETRAPHOSPHATE | | |
| PYROXAMIDE | 15 | HDAC1 inhibitor |
| PYRVINIUM PAMOATE | 13 | Antiparasitic/antihelminth |
| QUETIAPINE | 1 | Antipsychotic |
| QUINESTROL | 15 | Synthetic steroidal estrogen |
| R-547 | 18 | CDK 1, 2, 4 inhibitor |
| RALOXIFENE | 3 | Estrogen receptor modulator |
| RALTITREXED | 3 | Chemotherapeutic |
| REGORAFENIB | 9 | VEGFR2 and TIE2 dual inhibitor |
| RITANSERIN | 1 | Serotonin receptor antagonist, 5-HT receptor |
| ROLIPRAM | 1 | Phosphodiesterase-4 inhibitor |
| ROLOFYLLINE | 1 | Adenosine A1 receptor antagonist |
| ROSUVASTATIN CALCIUM | 9 | Statin |
| (Crestor) | | |
| RUXOLITINIB | 6 | JAK 1/2 inhibitor |
| SALINOMYCIN | 10 | Potassium ionophore/Fe sequestration, Wnt inhibitor |
| SANGUINARIUM CHLORIDE | 18 | Antibacterial: PP2C and MKP-1 |
| SAXAGLIPTIN | 1 | Dipeptidyl peptidase-4 (DPP-4) inhibitor (for type-2 diabetes) |
| SB-705498 | 1 | Capsaicin-mediated activation of TRPV1 receptors (TRPV1) |
| | | antagonist |
| SB743921 | 18 | Kinesin spindle protein inhibitor |
| SELAMECTIN | 18 | Macrocyclic lactone |
| SERATRODAST | 1 | Thromboxane A2 (TXA2) receptor (TP receptor) antagonist |
| | | (asthma) |
| SERDEMETAN | 12 | Blocks degradation of p53 by inhibiting HDM2 |
| SERTACONAZOLE | 2 | Antifungal |
| SGI-1776 | 14 | PIM1, 2, 3 kinase inhibitor |
| SIMVASTATIN | 6 | Statin |
| SIPATRIGINE | 1 | Ion channel: sodium, calcium, tandem pore-domain K(+) channels |
| | | TREK-1 and TRAAK inhibitor |
| SISOMICIN | 10 | Aminoglycoside antibiotic |
| SORAFENIB | 9 | Raf inhibitor/VEGFR-2/PDGFR inh |
| STROPHANTINE | 18 | Cardiac Glycoside |
| OCTAHYDRATE | | |
| SU-9516 | 1 | CDK 1, 2, 4 inhibitor (largely selective for cdk2) |
| SULCONAZOLE NITRATE | 5 | Antifungal cream |
| SULFADOXINE | 1 | Antiparasitic |
| SULFAMETHIZOLE | 18 | Antibacterial |
| SULOCTIDIL | 12 | Vasodilator with liver toxicity |
| SUNITINIB MALATE | 15 | VEGFR2, PDGFRb, c-KIT inhibitor |
| TACEDINALINE | 4 | Class I HDAC inhibitor (HDAC1, etc) |
| TALTOBULIN | 14 | Microtubules, chemotherapeutic |
| TANAPROGET | 5 | Progesterone receptor agonist |
| TANESPIMYCIN (17-AAG) | 18 | HSP90 inhibitor |
| TAXOL | 5 | Chemotherapeutic |
| TEBIPENEM PIVOXIL | 1 | Antibacterial |
| TECALCET | 1 | Type II calcimimetic agent, acting on parathyroid cell calcium |
| HYDROCHLORIDE | | receptor |
| TEMOZOLAMIDE | 14 | Chemotherapeutic |
| TENIPOSIDE | 10 | Chemotherapeutic |
| TERCONAZOLE | 7 | Antifungal for vaginal yeast infection |
| TESMILIFENE | 9 | Anti-estrogen binding site, antineoplastic drug and |
| HYDROCHLORIDE | | chemopotentiator |
| TETRACAINE | 12 | Ion channel: Calcium; local anesthetic |
| THIMEROSAL | 18 | Antiseptic, antifungal |
| THIOGUANINE | 14 | Chemotherapeutic, Purine analog |
| THIRAM | 4 | Antiparasitic, antifungal |
| THONZONIUM BROMIDE | 18 | Antibacterial, antifungal |
| TIBOLONE | 1 | Steroid: estrogenic, progestogenic |
| TICLOPIDINE | 1 | Antiplatelet, ADP receptor inhibitor |
| TILMICOSIN | 1 | Antibacterial, macrolide antibiotic |
| TILORONE | 10 | Antiviral/interferon-inducer |
| TIOCONAZOLE | 1 | Antifungal |
| TIOGUANINE (6-thioguanine) | 16 | Chemotherapeutic |
| TIPIFARNIB | 7 | Farnesyltransferase inhibitor |
| TOBRAMYCIN | 1 | Antibiotic, Antibacterial |
| TOFACETINIB | 4 | JAK 1, 3 inhibitor |
| TOLAZOLINE | 4 | Alpha adrenergic receptor antagonist |
| HYDROCHLORIDE | | |
| TOLCAPONE | 1 | Selective, potent and reversible nitrocatechol-type inhibitor of the |
| | | enzyme catechol-O-methyltransferase (COMT) |
| TOLONIUM CHLORIDE | 11 | Dye |
| TOPOTECAN | 13 | Chemotherapeutic |
| TREQUINSINçHCL | 1 | cGMP-inhibited phosphodiesterase (PDE3; IC50 = 250 pM) |
| | | inhibitor |
| TRIFLUOPERAZINE | 3 | Adrenoreceptor, dopamine receptor antagonist for schizophrenia |
| TRIFLURIDINE | 6 | Antiviral derivative of thymidine |
| TRIMEBUTINE | 1 | Antimuscarinic, mild opioid agonist |
| TRIMETHOBENZAMIDE | 1 | Antiemetic |
| HYDROCHLORIDE | | |
| TYLOSIN | 1 | Antibiotic, veterinary medicine |
| TYLOXAPOL | 8 | Blocks lipolytic activity |
| URACIL | 7 | Ribonucleoside, pyrimidine |
| URSODIOL | 2 | Bile acid used to treat gall stones |
| V-51 | 2 | Antipsychotic |
| VALACICLOVIR | 1 | Antiviral |
| VANDETANIB | 1 | VEGFR, EGFR, RET |
| VIGABATRIN | 13 | GABA transaminase inhibitor |
| VILAZODONE | 2 | Seratonin reuptake inhibitor |
| VILDAGLIPTIN | 1 | Dipeptidyl peptidase-4 (DPP-4) inhibitor |
| VINBLASTINE | 14 | Chemotherapeutic, microtubules |
| VINCRISTINE | 14 | Chemotherapeutic |
| VINDESINESULFATE | 9 | Chemotherapeutic, microtubules |
| VINFLUNINE TARTRATE | 14 | Chemotherapeutic, microtubules |
| VINORELBINE | 16 | Microtubules |
| VINPOCETINE | 1 | Nootropic (memory) |
| VORINOSTAT | 18 | HDAC inhibitor |
| VX-689 (MK5108) | 1 | Aurora A kinase inhibitor |
| XL019 | 3 | JAK2 inhibitor |
| XYLAZINE | 4 | Alpha2 adrenergic receptor agonist |
| Y-3642 | 1 | ROK inhibitor, NSAID |
| YM-155 | 18 | Survivin |
| ZINC PYRITHIONE | 9 | Antifungal/Antibacterial cream |
| ZIPRASIDONE | 3 | Dopamine/seratonin antagonist (treatment of schizophrenia |
| | | agitation) |

Compounds from Table 1 that exhibited >70% killing activity against all 18 relapsed AML cases tested were also identified. These drugs (52 compounds) were active, independent of the mutational status of the patient leukemia cells. Therefore, in some cases, AML patient samples can be tested for sensitivity to drugs, for example two or more drugs, selected from the 52 compounds set forth in Table 2.

TABLE 2

ABAMECTIN
ACRISORCIN
ADEFOVIR
ALEXIDINE
AMINACRINE
AT-7519
BECATECARIN
BMS-387032
BORTEZOMIB
CARFILZOMIB
CETYLPYRIDINIUM CHLORIDE
CLOFARABINE
CNF-2024
CYCLOHEXIMIDE
DARAPLADIB
DAUNORUBICIN
DIGITOXIN
DIGOXIGENIN
DIGOXIN
DORAMECTIN
DOXORUBICIN
EMETINE
EPIRUBICIN
FLURANDRENOLIDE
GENTIAN VIOLET
IDARUBICIN
IODOQUINOL
IVERMECTIN
JTC-801
LANATOSIDE C
LAQ-824
METHYLENE BLUE
MITOXANTRONE
OUABAIN
PF-04928473
PF-562271
PHA-767491
PHENYLMERCURIC ACETATE
PONATINIB
PROSCILLARIDIN
PUROMYCIN
R-547
SANGUINARIUM CHLORIDE
SB743921
SELAMECTIN
STROPHANTINE OCTAHYDRATE
SULFAMETHIZOLE
TANESPIMYCIN (17-AAG)
THIMEROSAL
THONZONIUM BROMIDE
VORINOSTAT
YM-155

In the present method any type of hematological cancer cells, such as leukemic cells, can be used. Leukemic cells useful in the method include, for example, AML, chronic myelogenous leukemia (CIVIL), acute lymphocytic leukemia (ALL), chronic lymphocytic leukemia (CLL), and multiple myeloma cells, depending on the type of leukemia the subject has. It should be noted that, throughout, leukemic cells and AML cells, in particular, are referenced as exemplary cancer cells, but various hematological cancer cells and various other cancer cells (e.g., ovarian cancer cells) could be used to identify compounds with growth inhibitory activity and/or cytotoxic activity against the cancer cells from a specific subject according to the methods disclosed herein.

Hematological cancer cells can be obtained from the subject by obtaining a sample of the subject's bone marrow, blood, or white blood cells using techniques known in the art. Other cancer cells, such as ovarian cancer cells, may be obtained by biopsy or acquisition of a solid tumor sample. In the methods provided herein, bone marrow cells can be obtained by bone marrow aspiration or biopsy using methods known in the art. See, for example, Bain, Bone marrow aspiration, J. Clin. Pathol. 54(9): 657-663 (2001). Peripheral blood leukemic cells can also be obtained using standard art-recognized methods. See, for example, Coustan-Smith et al., Use of peripheral blood instead of blood marrow to monitor residual disease in children with acute lymphoblastic leukemia, Blood 100: 2399-2402 (2002). The subject may be newly diagnosed with leukemia or other cancer or may have a leukemia or other cancer that is refractory to treatment.

As used herein, refractory or chemorefractory refers to the state of a target cell that does not shrink or vanish in response to an administered therapeutic agent, such as a chemotherapeutic drug or a molecularly targeted agent. The target cells may be refractory to the therapeutic agent right away or it may become refractory during treatment, as the cancer cells evolve to evade the effect of the therapeutic agent. It should be noted that refractory does not necessarily refer to an absolute response, such as no effect as compared to a control, but may refer to a reduced effectiveness over time or reduced sensitivity.

Optionally the cells are promptly frozen after collection from the subject to allow for storage and/or transport from the point of sample acquisition to the point of laboratory testing. If the cells are frozen and thawed for use in the subsequent steps of the method, freezing and thawing occur prior to the pre-culturing step, as some of the frozen cells will not survive the freeze-thaw step or may not survive culturing after the freeze-thaw step.

The pre-culturing step involves placing the obtained cells into culture medium in, for example, a plastic flask under culture conditions (e.g., using the same culture conditions used for the subsequent contacting step). The cells are maintained in culture for a period of time sufficient to allow cell death of a subset of the cells. By allowing cells to die in the pre-culture step, the method allows for elimination of cells adversely affected by previous steps (such as collection, storage, freezing and thawing, inability to adapt to the culture conditions, and the like). The period of time for pre-culturing is generally between at least 12 hours (e.g., 24-96 hours).

The pre-cultured cells are the sorted through a cell sorter (e.g. a fluorescence activated cell sorter (FACS)). Cells that do not survive the pre-culturing step can then be separated from the viable cells by selecting for viable cells in the sorting process. Similarly, non-cancer cells can be separated from the viable cancer cells to arrive at a relatively pure population of viable cancer cells from the subject. Eliminating non-viable and non-cancerous cells in the sample significantly reduces the false-positive results that arise from a drug killing or appearing to kill what can be a fairly high percentage of non-cancerous cells or non-viable cancer cells that are contaminating the sample. Thus, pre-culturing and sorting gives a population of test cells that best reflect the response of the cells in vivo, as those cells that react poorly to laboratory conditions such as freezing, thawing, storage, and culturing are eliminated and as non-cancer cells are eliminated. Upon cell sorting, a relatively pure population of viable cancer cells is obtained. As used herein, relatively pure means at least about 95% viable cancer cells in the population, at least 98% viable cancer cells in the population, or at least about 99% viable cancer cells in the population. A relatively pure population of viable cancer cells, as used herein, includes a 100% pure population of viable cancer cells. As used herein, essentially pure means at least 98% pure viable cancer cells. Thus, when reference is made to a relatively pure population of cells, it is understood that essentially pure populations of cells are contemplated.

Aliquots of the viable cells are then dispensed into individual wells of one or more multi-well tissue culture plates. In the methods provided herein, the multi-well tissue culture plate can be a 6-, 12-, 24-, 48-, 96-, 384-, 1536-, or 3,072-well dish. In the methods provided herein, cells can be dispensed into two or more, 10 or more, 20 or more, 30 or more, 40 or more, 50 or more, 60 or more, 70 or more, 80 or more, 90 or more, 100 or more, 200 or more, 300 or more, 400 or more, 500 or more, 600 or more, 700 or more, 800 or more, 900 or more, 1000 or more, 1100 or more, 1200 or more, 1300 or more, 1400 or more, 1500 or more, or 3000 or more individual wells of a multi-well tissue culture plate. The cells are optionally dispensed with an automated cell dispensing unit. The number of cells dispensed into each well can vary depending on the size of the well and the cell density and number of cells in a sample volume, but the number of viable cells per well in a plate or set of plates used in the method will be consistent across wells. By way of example, about 4,000 to about 5,000 viable cells can be dispensed into each well of the 1,536-well plate. One of skill in the art can identify the optimal cell density to ensure viability and cell growth during the culture period.

The cells are cultured in the one or more multi-well plates for a period of time, optionally in the same type of media and same culture conditions used in the pre-culturing step. This period of time provides the cells an opportunity to acclimate to the culture conditions and grow but is not so long as to stress the cultured cells. Generally this culture period can be for a period of less than 60 minutes, 1-24 hours, or up to 2-5 days. Standard culture conditions (e.g., in a humidified incubator at appropriate CO₂ levels) are used and are known to one of skill in the art.

The cultured cells are then contacted with one or more of the compounds of Table 1 or Table 2. The amount of compound in each well can vary. By way of example, a series of dilutions of each compound can be used, such that one well receives a given dilution of a compound and another well gets a second dilution of the same compound. Alternatively, an IC50 or other dose of each compound can be selected for testing in a single assay.

The cells within each individual well are contacted with at least one compound, although a subsets of wells can serve as untreated controls and a subset can serve as duplicates, triplicates, or the like if enough wells with viable cells are available. This format gives an opportunity to test numerous compounds with one or more multi-well plates. If combinations of compounds are tested, two or more compounds from Table 1 and/or 2 can be added to the same well. Such combinations of compounds could be used in parallel with single compounds from Table 1 and/or 2, but could also be contacted in subsequent assays. By way of example, at least 52 wells of cells, each contacted with a unique compound, may be used. These 52 wells can be duplicated to provide an N of 2 for each compound; the 52 wells could be provided in triplicate to provide an N or 3, and so forth. Optional controls include a select number of wells that comprise viable cells untreated with any of the compounds and of wells comprising cells treated with a reagent that results in cell death.

The period of time the cells of the multi-well plate are incubated with the compounds can vary. For example, the cells can be cultured in contact with the compound for about 60 to about 120 hours. For example, the cells can be cultured for about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96 hours. Optionally, the contacting can occur for about 1 to about 5 days, for example, about 3 days.

Following the contacting step, the wells are tested to identify which wells and which corresponding compounds (or combination thereof) show inhibition of cell growth or promoted cytotoxity, as compared to a control (e.g. an untreated control well or set thereof). The number of viable cells can be counted using a variety of techniques, including the use of commercially available reagents like CellTiter-Glo (Promega, Madison Wis.) or AlamarBlue (Thermo Fisher Scientific, Waltham, Mass.). Fluorescent stains can also be used to quantify viable cell numbers using flow cytometry (FACS).

Methods for Identifying One or More Compounds that Sensitize Refractory Cancer Cells from a Specific Subject

Provided herein is a method of rapidly identifying compounds that sensitize a subject's refractory cancer cells, such as hematological cancer cells (e.g., leukemic cells, such as AML cells) or ovarian cancer cells that are refractory to a therapeutic agent. As used herein, therapeutic agent refers to drugs or molecularly targeted agents used to treat cancer. As used herein, sensitization or sensitizing refers to promoting sensitivity in cells that showed reduced or no sensitivity or restoring sensitivity to cells that show reduced or no sensitivity after a period of sensitivity to treatment with the therapeutic agent.

The method for identifying one or more compounds that sensitize refractory hematological or ovarian cancer cells from a subject comprises the steps of obtaining hematological cancer cells from bone marrow or peripheral blood cells from a subject having a hematological cancer or ovarian cancer cells from a subject with ovarian cancer, wherein the hematological or ovarian cancer cells are refractory (or resistant) to a therapeutic agent.

These cells are optionally frozen for subsequent use. Freezing may be necessary when a first assay is performed to identify a low dose of the therapeutic agent to be used in the subsequent assay of identifying one or more compounds that sensitize refractory cells, as the time to run the first assay and identify the low dose of the therapeutic agent could compromise the results of the second assay using the low dose of the therapeutic agent to identify sensitizing compounds. Thus, in certain embodiments of the method, a first and second portion of the frozen cells are thawed, pre-cultured, and sorted as described above to obtain a relatively pure population of viable cancer cells for use in subsequent steps of the method.

Optionally, a first assay is performed to determine a low dose of the therapeutic agent for use in the sensitization method. Aliquots of the cells are dispensed into individual wells of one or more multi-well tissue culture plates and the dispensed cells in each well are contacted with a concentration of the therapeutic agent. To this end, determining which concentrations of the therapeutic agent inhibit cell growth or promote cytotoxicity to determine the IC10 of the therapeutic agent. The cells in a set of the wells is contacted with a first concentration, a second concentration, a third concentration, or a fourth concentration (up to at least ten concentrations) of the therapeutic agent, although additional concentrations may also be used to develop a dose-response curve for the therapeutic agent. The concentration of the therapeutic agent is selected by determining the inhibition of cell growth or promotion of cytotoxicity.

In another assay, optionally using cells obtained in the same step as the cells used for determining the low dose of the therapeutic agent, a second portion of the frozen cells are thawed, pre-cultured, and sorted as described above to obtain a relatively pure population of viable cancer cells for use in subsequent steps. The method further comprises dispensing aliquots of the cells into individual wells of one or more multi-well tissue culture plates and contacting the cells in a first subset of the wells with a combination of a low dose of the therapeutic agent to which the cells are refractory and a selected dose of a unique compound selected from the compounds set forth in Table 1 or Table 2, wherein the selected dose of each compound varies across wells in the first subset. The assay also includes contacting the cells in a second subset of the wells with the same compounds and selected doses thereof of the first subset of wells. One or more compounds that sensitize the cells to the therapeutic agent are then identified by determining which compounds inhibit cell growth or promote cytotoxicity in the first subset of wells of the contacted cells more than the same one or more compounds in the second subset of wells. In this assay, the low dose is optionally the IC₁₀ of the therapeutic agent in inhibiting cell growth or promoting cytotoxicity of the hematological cancer cells from bone marrow or peripheral blood cells from the subject having a hematological cancer or ovarian cancer cells from the subject with ovarian cancer. Optionally, the selected low dose is the IC₁₀ of the therapeutic agent as determined using the assay steps described above. Identifying one or more compounds that sensitize the cells to the therapeutic agent involves determining whether the IC50 of the compound is reduced in the presence of the low dose of the therapeutic agent as compared to the absence of the therapeutic agent.

Optionally the multi-well tissue culture plate as used in the method is a 1,536 well tissue culture plate. Optionally, about 4,000 to about 5,000 viable cells are dispensed into each well.

As used herein, low dose of the therapeutic agent is meant, for each therapeutic agent, a dose of about IC₅-IC₂₅, about IC₅-IC₁₅, or about IC₁₀, The method optionally includes performing an eight-point or ten point dose-response screen (i.e., 8-10 doses of each therapeutic agent are plated into a well in the multi-well plate and a dose-response curve is generated) to identify the low dose of the therapeutic agent (e.g., IC₁₀).

The viable cancer cells in a second subset of wells are contacted with a unique compound selected from the compounds set forth in Table 1 or Table 2 but without the therapeutic agent. Thus, the first subset is contacted with a combination of at least one compound and the therapeutic agent, the second subset is contacted with the compound or compounds (i.e, the same compound or compounds as contacting the first subset). Any subset can be, for example, 52 wells, 412 wells, or the like. Optionally the first subset is a 1,536-well plate and the second subset is a 1,536-well plate, optionally each plate including one or more sets of controls. The method optionally includes performing an eight-point or ten-point dose-response screen (i.e., 8-10 doses of each compound with and without the low dose of the therapeutic agent are plated into a well in the multi-well plate and a dose-response curve is generated) to identify any shift in the IC₅₀ for the compound in the presence of the low dose of the therapeutic agent as compared to the absence of the therapeutic agent. For example, in a first subset of wells, the cancer cells are incubated with the low dose (e.g., IC₁₀) of the therapeutic agent to which the cancer cells are resistant along with the compounds at varying dilutions, whereas the second subset includes only the compounds at varying dilutions. The results of the combination screen determine whether the survival curve for any of the compounds shifts to the left (i.e., toward a lower IC₅₀ value) when the low dose of the therapeutic agent to which the cells are resistant is also present in the wells as compared to the wells lacking the therapeutic agent.

It is understood that each multi-well plate can comprise replicates of each well contacted with a unique compound and/or unique dose. Additionally, it is understood that each multi-well plate optionally includes control wells (e.g., containing cells that are not contacted with either a compound or a therapeutic agent).

Methods of Treating a Subject with Cancer

Upon identification of one or more compounds from Table 1 or Table 2 that inhibit growth of, promote cytotoxicity in, or sensitize cancer cells in the subject's sample using any one of the methods described herein, the subject can be treated with the one or more compounds, optionally in combination with a therapeutic agent to which the cells are refractory in the absence of the one or more identified compounds.

For example, provided herein is a method for treating acute myeloid leukemia (AML) in a subject comprising obtaining bone marrow or peripheral blood leukemic cells from a subject having AML; pre-culturing the cells as described above (optionally after freezing and thawing); sorting the cells to achieve a relatively pure population of viable cancer cells. dispensing aliquots of the cells into individual wells of a multi-well tissue culture plate as described above; culturing the cells in each well and contacting the cells in each well with at least one compound selected from the compounds set forth in Table 1 or Table 2 as described above or a compound and a therapeutic agent to which the cells are refractory; identifying one or more compounds that inhibit cell growth, promote cell death, or sensitize the contacted cells; and administering an effective amount of the one or more identified compounds, optionally along with one or more therapeutic agents or anti-cancer therapies. Optionally the subject has leukemia (e.g., AML, CML, ALL, chronic lymphocytic leukemia (CLL), and multiple myeloma) or ovarian cancer.

As used throughout, by subject is meant an individual. For example, the subject is a mammal, such as a primate, and, more specifically, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations are contemplated herein. The term does not denote a particular age or sex. Thus, adult and newborn subjects, whether male or female, are intended to be covered. As used herein, patient or subject may be used interchangeably and can refer to a subject afflicted with a disease or disorder (i.e., AML).

As used throughout, AML is a cancer of the myeloid lineage of blood cells, characterized by the rapid growth of abnormal cells that build up in the bone marrow and blood and interfere with the function of normal blood cells and other organ systems. In this aggressive disease, excess myeloblasts (immature white blood cells that are not lymphoblasts) are found in the bone marrow and blood and are frequently disseminated in other tissues. Acute myeloid leukemia is also called acme Myeloblastic leukemia, acute myelogenous leukemia, or acute nonlymphocytic leukemia.

In the methods provided herein, the subject can have a refractory cancer (such as a chemorefractory AML). For example, a subject with refractory AML, does not respond to or does not achieve complete remission with a course of treatment. For example, a subject has refractory AML if the subject has a remaining myeloblast count of 5% or more after one or two cycles of intense remission induction therapy, for example, chemotherapy. The most common remission induction regimens for AML include administration of cytarabine, most often given continuously for seven days through an intravenous (IV) line. An anthracycline drug, such as daunorubicin or idarubicin, is also given in a single IV dose on each of three days during the first week of treatment. For people whose AML has a mutation in the FLT3 gene, midostaurin or other agent that specifically targets cells with a specific FLT3 mutation, may be added. Thus provided herein is a method of treating AML in a subject comprising administering to the subject midostaurin or other agent that specifically targets cells with a specific FLT3 mutation, wherein the AML cancer cells of the subject comprise a FLT3 mutation. The method optionally further comprises identifying the mutation in the AML cancer cells of the subject.

Also provided is a method of treating AML in a subject by administering to the subject one or more compounds of Table 1. Optionally, the method further comprises identifying mutations in one or more genes of the AML cancer cells prior to administration.

In the methods provided herein, the subject can have recurrent or relapsed cancer (e.g., relapsed AML). Subjects who achieve a complete remission to initial treatment and then experience a cancer recurrence are said to have relapsed cancer. Relapse of leukemia, for example, may occur within days, months, or years after the initial remission. In many cases, relapse occurs within the first two years of initial treatment.

Examples of pathway or selective protein-targeted inhibitors include, but are not limited to, inhibitors that target a specific protein or pathway that is mutated or altered specifically in the cancer patient sample based on DNA sequencing analysis of the patient tumor DNA. Examples in the context of AML include inhibitors that target AML, cells with a mutated FLT3 gene, for example, an FLT3-ITD mutation or a FLT3-TKD mutation. In some examples, harmine is administered to a patient having a FLT3-ITD mutation, either alone or in combination with another compound from Table 1 or Table 2, or in combination with a second anti-leukemic therapy. In some methods, an inhibitor that targets AML cells with a mutated FLT3 gene can be administered with an inhibitor that targets JAK2 and/or an inhibitor that targets JAK3.

Throughout, treat, treating, and treatment refer to a method of reducing or delaying one or more effects or symptoms of AML. The subject can be a subject newly diagnosed with AML that has not undergone treatment for AML or a subject diagnosed with refractory or relapsed AML after initial treatment, for example, after induction chemotherapy. Treatment can also refer to a method of reducing the underlying pathology rather than just the symptoms. The effect of the administration to the subject can have the effect of but is not limited to reducing one or more symptoms of AML, a reduction in the severity of AML, or the complete ablation of AML, or a delay in the onset or worsening of AML. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between.

Administration can be carried out using therapeutically effective amounts of one or more compounds described herein for periods of time effective to treat cancer. In some methods, the one or more compounds treat AML and reduce the recurrence of AML. By reducing the recurrence of AML is meant a method of preventing, precluding, delaying, averting, obviating, forestalling, stopping, or hindering the onset, incidence or severity of the reappearance of AML in a subject.

In some methods, one or more compounds selected from the group consisting of an HSP90 inhibitor, a cardiac glycoside, an HDAC inhibitor, an ion channel inhibitor, a calcium ion channel blocker, a statin, a CDK inhibitor, a proteasome inhibitor, a WNT inhibitor, a macrocyclic lactone, a lipase inhibitor, an antiparasitic, an antifungal and an antibiotic are administered to the subject.

In some methods, a statin selected from the group consisting of pitavastatin, atorvastatin calcium, fluvastatin, rosuvastatin, mevastatin, cerivastatin and simvastatin is administered to the subject. In some methods pitavastatin is administered to the subject. In some methods, pitavastatin is administered in combination with apilimod mesylate to the subject.

In some methods, an HSP90 inhibitor selected from the group consisting of CNF-2024, PF-04928473 and HSP-990 is administered to the subject. In some methods, a cardiac glycoside selected from the group consisting of digitoxin, digoxigenin, digoxin, lanatoside C, proscillaridin and ouabain is administered to the subject.

In some methods, an HDAC inhibitor selected from the group consisting of vorinostat, LAQ-824, pyroxamide and bufexamac is administered to the subject. In some methods, an ion channel inhibitor selected from the group consisting of dronedarone, salinomycin and lasalocid sodium is administered to the subject.

In some methods, a calcium ion channel blocker selected from the group consisting of niguldipine, tetracaine HCl, amlodipine, lomerizine HCl, azelnidipine and manidipine is administered to the subject.

In some methods, a CDK inhibitor selected from the group consisting of AT-7519, AZD-5438, BMS-387032, PHA-767491, PHA-793887, R-547 and PHA-690509 is administered to the subject. In some methods, a proteasome inhibitor selected from the group consisting of bortezomib and carfilzomib is administered to the subject.

In some methods, a WNT inhibitor selected from the group consisting of ivermectin and salinomycin is administered to the subject. In some methods, a macrocylic lactone selected from the group consisting of ivermectin, abamectin, doramectin and selemectin is administered to the subject. In some methods, ivermectin is administered in a formulation that reduces the ability of ivermectin to cross the blood-brain barrier.

In some methods, a lipase inhibitor selected from the group consisting of darapladib and orlistat is administered to the subject.

In some methods, an antiparasitic selected from the group consisting of mebendazole, primaquine diphosphate, pyrvinium pamoate, iodoquinol, hycanthone, artesunate, clioquinol, dequalinium chloride and narasin is administered to the subject.

In some methods, an antibiotic selected from the group consisting of sulfamethizole, cetylpyridinium chloride, tanespimycin, gramicidin and sisomicin is administered to the subject.

According to the methods disclosed herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

The effective amount may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day.

Any appropriate route of administration may be employed, for example, parenteral, intravenous, subcutaneous, intramuscular, intraventricular, intracorporeal, intraperitoneal or oral administration. Administration can be systemic or local. Multiple administrations and/or dosages can also be used. Effective doses can be extrapolated from dose-response curves derived from in vitro drug sensitivity testing or animal model test systems. The disclosure also provides a pharmaceutical pack or kit comprising one or more containers filled with one or more of the ingredients of the pharmaceutical compositions. Instructions for use of the composition can also be included. Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed. Publications cited herein and the material for which they are cited are hereby specifically incorporated by reference in their entireties.

In the methods provided herein, one or more compounds from Table 1 or Table 2 are administered to the subject with cancer (e.g., a subject having AML). Therefore, also provided is a method of treating AML, in a subject comprising administering to the subject having AML, an effective amount of one or more compounds set forth in Table 1 or Table 2. It is understood that any of the compounds set forth in Table 1 or Table 2 can be administered in combination with another compound set forth in Table 1 or Table 2 to increase toxicity to AML cells. A compound from Table 1 or Table 2 can be administered prior to, concurrently with, serially or after administration of a different compound from Table 1 or Table 2 or a therapeutic agent to which the cancer cells were refractory in the absence of the one or more compounds.

Any of the methods provided herein can optionally further include administration of a second anti-cancer therapy (e.g., an anti-leukemic therapy) to the subject. In the methods provided herein, for example, the second anti-leukemic therapy can be chemotherapy, molecular targeting therapy, immunotherapy, radiotherapy, a pathway or selective protein-targeted inhibitor, or a bone marrow transplant. In some methods, one or more anti-leukemic therapies selected from the group consisting of chemotherapy, molecular targeting therapy, immunotherapy, radiotherapy, a pathway or selective protein-targeted inhibitor, or a bone marrow transplant are administered to the subject. The second anti-leukemic therapy can be administered prior to, concurrently with, serially or after administration of one or more compounds from Table 1 or Table 2.

Examples of chemotherapeutic agents include, but are not limited to, cytarabine, daunorubicin, idarubicin, mitoxantrone, cladribine, fludarabine, topotecan, etoposide, 6-thioguanine, hydroxyurea, corticosteroid drugs (for example prednisone or dexamethasone), methotrexate, 6-mercaptopurine, azacitidine and decitabine.

A number of embodiments have been described. Nevertheless, it will be understood that various modifications may be made. Accordingly, other embodiments are within the scope of the following claims.

### Examples

### Drug Sensitivity Assay

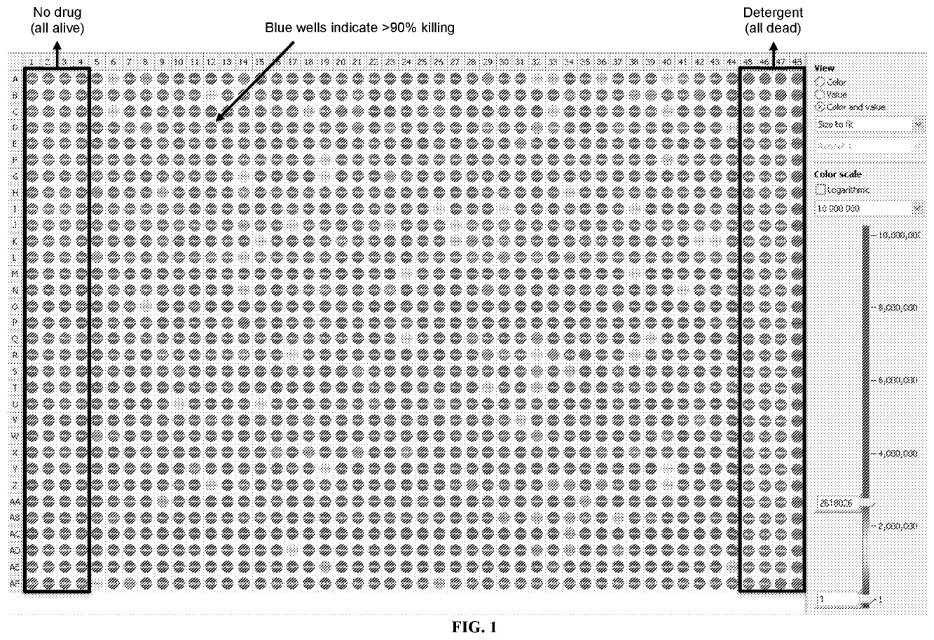

Drug sensitivity assays were performed using a 1,536-well platform where all cells and reagents were deposited in wells using an acoustic liquid handling unit that is highly accurate for dispensing of nanoliter volumes of liquid. Cell density was first optimized in pilot experiments and was found to be optimal between about 800,000-900,000 cells/ml (4,000-4,500 cells in the total 5 microliter volume of the well). After dispensing the cells into the wells, an independent drug at a known concentration is added to each well and incubated with the cells. After three days, the percentage of live cells in each well is determined to assess the killing efficiency of each compound. Results using 18 independent patient samples to date has identified 412 compounds that can kill over 70% of chemorefractory AML cells (“blue” wells in FIG. 1) after a 3-day culture period where cells are incubated with individual drugs. Cell viability assays are known to those of skill in the art. See, for example, Pemovska et al., Individualized systems medicine strategy to tailor treatments for patients with chemorefractory acute myeloid leukemia. Cancer Discovery 3:1416-1429, 2013; Pabst et al., Identification of small molecules that support human leukemia stem cell activity ex vivo. Nature Methods 11:436-442, 2014; Baccelli et al., A novel approach for the identification of efficient combination therapies in primary human acute myeloid leukemia specimens. Blood Cancer Journal 7:e529, 2017. In studies described herein, the 1,536-well format was utilized, which allowed for use of four times fewer cells as well as less drug and media components due to the small assay volume of 5 microliters. This approach gave highly reproducible results after plating of cells and culturing at 37° C., at 5% CO₂, for a total of 3 days. The reproducibility of these conditions to support leukemia cell growth was confirmed, as shown in FIG. 1 (columns 1-4), where about 100% viability of cells was observed after 3 days of culture in 128 independent wells that received no compound. Cell viability was measured using the cell permeable dye alamarBlue (resazurin), which is reduced to the fluorescent compound resorufin through the activity of redox enzymes in metabolically active cells (Thermo Fisher, Waltham, Mass.). Other compounds like CellTiter-Glo (Promega, Madison, Wis.) are equally appropriate reagents for determining cell viability.

### Media Conditions for Culturing of Human Bone Marrow or Peripheral Blood Leukemic Cells

Conditions for the drug screening assays were as described in Pabst et al., Identification of small molecules that support human leukemia stem cell activity ex vivo, Nature Methods 11:436-442, 2014). Using this protocol, cells were cultured in Iscove's Modified Dulbecco's Medium (IMDM), 15% BIT (bovine serum albumin, insulin, transferrin; Stem Cell Technologies 09500 (Vancouver, Calif.)), 100 ng/ml SCF (Shenandoah 100-04 (Warwick, Pa.)), 50 ng/ml FLT3L (Shenandoah 100-21), 20 ng/ml IL-3 (Shenandoah 100-80), 20 ng/ml G-CSF (Shenandoah 100-72), 10-4 M β-mercaptoethanol, gentamicin (50 μg/ml) and ciprofloxacin (10 μg/ml). The media recipe is shown in Table 3.

TABLE 3

| | | | Stock | | Dilution | Final | |
|---|---|---|---|---|---|---|---|
| Media recipe | Volume | | Concentration | | Factor | Concentration | |
| StemSpan ™ Media (Stem Cell | 10 | mL | — | | — | — | |
| Technologies) | | | | | | | |
| Human Stem Cell Factor (SCF) | 10 | μL | 100 | μg/mL | 1,000x | 100 | ng/mL |
| Human Flt3 Ligand (FLT3L) | 5 | μL | 100 | μg/mL | 2,000x | 50 | ng/mL |
| Human Recombinant IL-3 (IL-3) | 2 | μL | 100 | μg/mL | 5,000x | 20 | ng/mL |
| Human Recombinant | 2 | μL | 100 | μg/mL | 5,000x | 20 | ng/mL |
| Granulocyte Colony Stimulating | | | | | | | |
| Factor (G-CSF) | | | | | | | |
| StemRegenin-1 (SR1) (Stem | 1 | μL | 10 | mM | 10,000x | 1 | μM |
| Cell Technologies) | | | | | | | |
| UM729 (Stem Cell | 10 | μL | 954 | μM | 1,000x | 954 | nM |
| Technologies) | | | | | | | |

### Protocol for Thawing Frozen Human Bone Marrow Cells and Overnight Culture

Cells were removed from liquid nitrogen and placed in a 37° C. bath, just until thawed. For 1,536-well screens utilizing 6,000 wells about 100 million cells were thawed. The volume was doubled with pre-warmed culture media and placed on ice for about one to two minutes. The cells were brought to a final volume of about 8 to 10 mL before spinning down at 1,100 rpm for 5 min at 10° C. The pelleted cells were resuspended in 10 mL of culture media and plated in a tissue-culture treated T75 flask at a density of 1 to 2 million cells/ml. The cells were cultured overnight prior to fluorescence-activated cell sorting of viable cells on the following day.

### Preparation of Human Bone Marrow Cells

After cells were cultured overnight, a cell scraper was used to remove any cells that adhered to the plastic. The cells were gently pipetted up and down to break up aggregates. Then, the entire volume was transferred to a 50 mL conical tube through a 70 micron cell strainer to remove cell clumps. The T75 flask was rinsed and scraped with about 10 mL HBSS and transferred to the same 50 mL conical tube through the strainer. The harvested cells were spun down at 1,100 rpm for 5 min at 10° C. The cells were then resuspendd in about 300-500 microliters FACS buffer (HBSS+2% FBS+1× propidium iodide) and transferred to a FACS tube. The tube was placed on ice and viable cells that are forward- and side-scatter gated on viable myeloid blasts were sorted by FACS (the scatter gate should exclude small lymphocytes, large myeloid cells, and debris). After sorting the required number of viable cells, the cells were spun down at 1,100 rpm for 5 min at 10° C. and subsequently resuspended at 4,000-4,500 cells per 5 microliter volume of complete culture media with growth factors and penicillin/streptomycin. Using frozen cells, overnight culture, and flow cytometry ensured that there were 100% viable cells plated in the 1,536-well plates, which greatly reduced the signal-noise inherent in using freshly isolated cells where a certain percentage of cells will die during adaptation to growth in plastic.

Identification of Approved Drugs with Cytotoxic Activity Against AML

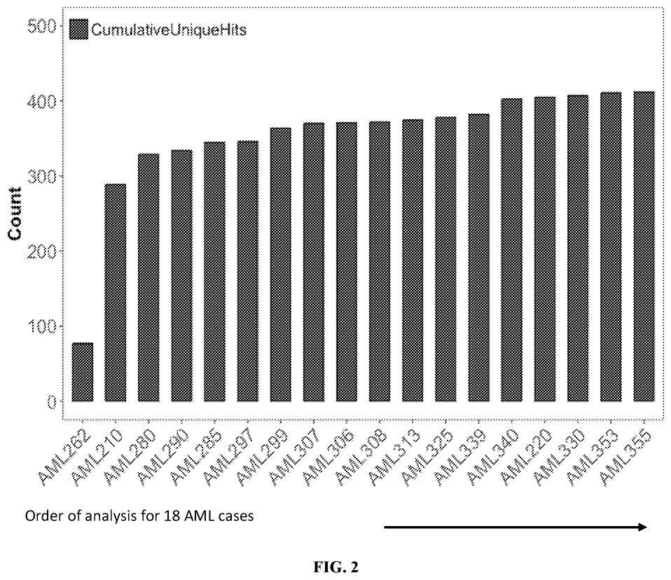

The first case of relapsed acute myeloid leukemia screened against the comprehensive set of 2,174 approved drugs was AML262. Some of the same drugs were active against the second case screened (AML210) and additional unique drugs with cytotoxic activity were identified. Although there will likely be additional unique drugs that are identified as more AML cases are evaluated, FIG. 2 shows that after evaluation of a total of 18 AML cases, saturation for identification of all FDA- (and other) approved drugs with activity against relapsed AML (412 total compounds)(Table 1) was approached. These 412 compounds have the ability to kill >70% of relapsed AML cells in a 3-day screen when used at a 10 μM concentration, as described above.

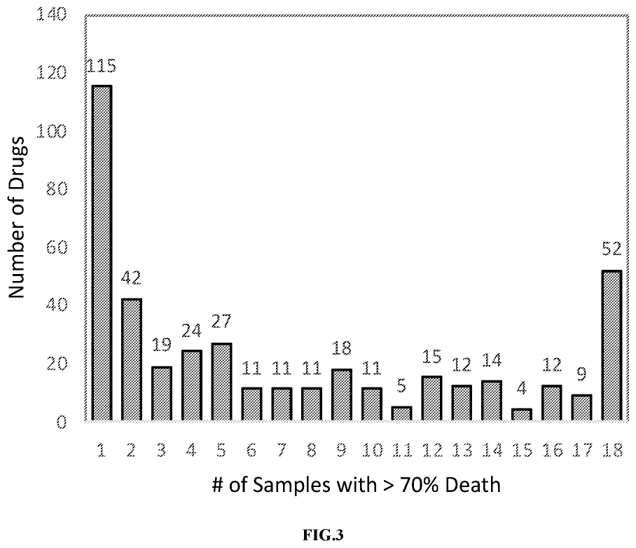

As shown in FIG. 3, there were a number of drugs that were active against the total number of AML cases. For instance, 52 drugs were active against all 18 cases, while 115 drugs were active in 1 out of 18 patient samples. The number of drugs active against varying numbers of AML cases is shown in FIG. 3. These results show that the majority of identified drugs have patient-specific cytotoxic activity.

Classes of Compounds with Cytotoxic Activity Against Relapsed and Refractory AML

Several classes of compounds that have cytotoxic activity against relapsed and refractory AML were identified. Examples of drugs within each class that have significant cytotoxic activity are listed below in Table 4. This is not a complete list of all drugs within each class or a complete list of classes. Patient-specific responses to specific classes of chemotherapeutic agents were observed. For instance, the taxols were highly active against 5 specific AML cases (out of 18 tested) and were not active against other cases. These results show the value of including drugs representing distinct classes of chemotherapeutic agents in the comprehensive drug sensitivity assay since some patients who are resistant to the standard-of-care chemotherapeutic agents for AML may be highly sensitive to other classes of chemotherapeutic agents. Drugs targeting specific receptor tyrosine kinases (RTK) or broad RTK inhibitors are not listed below but are included in the 412 drug list when these had cytotoxic activity.

TABLE 4

Cardiac Glycosides
Digitoxin
Digoxigenin
Digoxin
Lanatoside C
Proscillaridin
Ouabain
HSP90 inhibitors
CNF-2024
PF-04928473
HSP-990
HDAC inhibitors
Vorinostat (SAHA)
LAQ-824
Pyroxamide
Bufexamac
Ion channel inhibitors
Dronedarone
Salinomycin
Lasalocid sodium
Calcium ion channel blockers
Niguldipine
Tetracaine HCl
Amlodipine
Lomerizine HCl
Azelnidipine
Manidipine
Statins
Pitavastatin calcium
Atorvastatin calcium
Fluvastatin
Rosuvastatin
Mevastatin
Cerivastatin
Simvastatin
CDK inhibitors
AT-7519
AZD-5438
BMS-387032
PHA-767491
PHA-793887
R-547
PHA-690509
Proteasome inhibitors
Bortezomib
Carfilzomib
WNT inhibitors
Ivermectin
Salinomycin
Macrocyclic lactones
Ivermectin
Abamectin
Doramectin
Selemectin
Lipase inhibitors
Darapladib
Orlistat
Antiparasitics
Mebendazole
Primaquine diphosphate
Pyrvinium Pamoate
Iodoquinol
Hycanthone
Artesunate
Clioquinol
Dequalinium chloride
Narasin
Antifungals
Acrisorcin
Ciclopirox ethanolamine
Itraconazole HCl
Benzethonium chloride
Piroctone Olamine
Posaconazole
Sulconazole nitrate
Ciclopirox
Antibiotics
Sulfamethizole
Cetylpyridinium chloride
Tanespimycin
Gramicidin
Sisomicin

### Identification of Best-in-Class Compounds

Ten AML patient samples (right 10 columns: AML 280, AML 285, etc.) were evaluated for sensitivity to individual statins using a 10-point dose-response drug screen where each drug was evaluated over a 1,000-fold concentration range against each patient sample shown. IC₅₀ values in μM (dose of drug that kills 50% of cells in the 3-day drug sensitivity assay) are shown below each patient number in Table 5 (light blue values indicate <1 μM, while purple boxes highlight an IC₅₀ value between 1-5 μM). Three out of 10 cases (285, 340, and 290) were highly sensitivity to statin-mediated killing, with the most significant activity observed using pitavastatin, atorvastatin, or fluvastatin. Two cases, AML 285 and AML 290, had IC₅₀ values at or below the plasma concentration (Cₘₐₓ) of pitavastatin and fluvastatin that is achievable in vivo at the FDA-recommended dose and schedule, suggesting that leukemic blasts within these two patients would have been killed using safe (approved) doses of pitavastatin or fluvastatin. Based on these findings, pitavastatin, alone or in combination with other compounds can be used for treatment of newly diagnosed or relapsed AML.

TABLE 5

| | In vivo | | | | | | | | | | |
|---|---|---|---|---|---|---|---|---|---|---|---|
| | cMax | AML | AML | AML | AML | AML | AML | AML | AML | AML | AML |
| Statin | (μM) | 280 | 285 | 339 | 340 | 290 | 297 | 299 | 307 | 330 | 343 |
| Pitavastatin calcium* | 0.25 | 1.349 | 0.157 | 5.24 | 0.475 | 0.149 | >10.0 | 1.814 | 0.726 | 9.831 | >10.0 |
| Atorvastatin calcium** | 0.01 | 2.241 | 0.475 | 9.116 | 0.732 | 0.584 | >10.0 | 2.901 | 1.09 | >10.0 | >10.0 |
| Fluvastatin sodium*** | 0.49 | 2.418 | 0.426 | 7.789 | 0.721 | 0.417 | >10.0 | 3.809 | 1.576 | >10.0 | >10.0 |
| Rosuvastatin calcium | | 8.349 | 1.473 | >10.0 | 2.516 | 1.462 | >10.0 | >10.0 | 3.787 | >10.0 | >10.0 |
| Lovastatin | | >10.0 | 3.808 | >10.0 | 8.92 | 4.405 | >10.0 | >10.0 | >10.0 | >10.0 | >10.0 |
| Pravastatin sodium | | >10.0 | >10.0 | >10.0 | >10.0 | >10.0 | >10.0 | >10.0 | >10.0 | >10.0 | >10.0 |
| PravastatinLactone | | >10.0 | >10.0 | >10.0 | >10.0 | 8.328 | >10.0 | >10.0 | >10.0 | >10.0 | >10.0 |
| Simvastatin | | | >10.0 | 5.077 | >10.0 | >10.0 | 6.943 | >10.0 | >10.0 | >10.0 | >10.0 |

Assay to Identify Compounds that Sensitize Cancer Cells to Agents to which the Cells are Refractory

Leukemic cells were acquired from patients with AML and were frozen and thawed as described above. For the first step, a sufficient number of cells was thawed, cultured overnight in the same growth media used to perform the subsequent drug sensitivity assay, sorted as described above to obtain a pure population of leukemic cells and then plated using a highly accurate Echo acoustic liquid handler into wells of a 1,536-well plate. Approximately 4,000 cells were plated per well. The therapeutic agent to which patient cells were clinically resistant was then added robotically to 8 wells in doses covering a 10,000-fold concentration range (from 1 nM to 10 μM) in order to identify the low dose of the single drug (for example, the IC₁₀) for the second step below. Triplicate 8-point dose-response platings of drugs with cells was performed as replicate controls. Cells were cultured with drug for a total of 3 days, after which time the percentage of viable cells ware determined in each well by addition of the alamarBlue substrate.

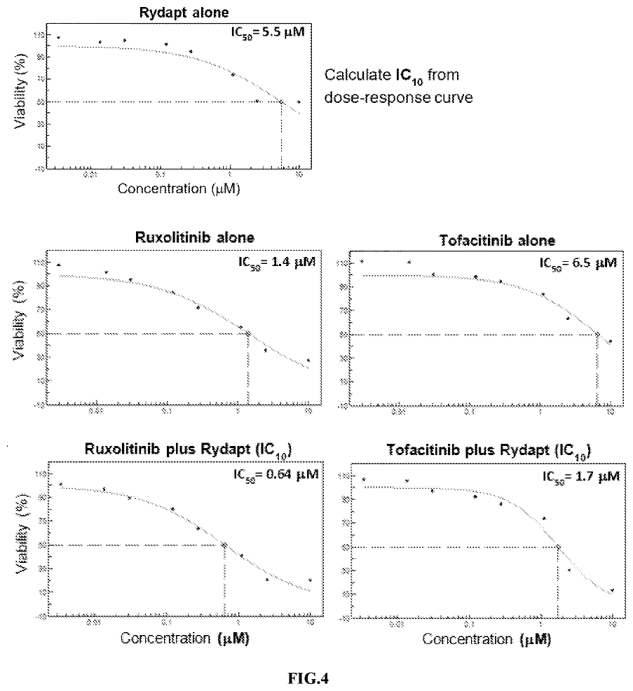

Once the IC₁₀ was determined for the specific therapeutic agent of interest in the first step, the second step was performed by setting up two identical sets of plates that include wells with the refractory leukemia patient cells. For this step, a large number of frozen leukemic cells were thawed, pre-cultured, and then sorted to allow sufficient numbers of cells for screening of multiple drug combinations. Cells were plated at a density of approximately 4,000 cells per well using the Echo into duplicate sets of 1,536-well dishes. In both sets of plates, drugs selected from the compounds set forth in Table 1 or Table 2 were added as single agents using an 8-point dose-response format for each drug (each drug is plated into 8 wells with varying dilutions of drug so that an IC₅₀ can be calculated from the dose-response curve for all of the compounds being tested). In one of the two sets of plates, all wells also received the low dose (for example, the IC₁₀) of the specific therapeutic agent that was evaluated in step 1 above). Cells are then incubated in a humidified tissue culture incubator at 37° C./5% CO₂ for 3 days and then viable cells were quantified using alamarBlue. Multiple control wells (a total of 128) that received cells cultured in the absence of drug were used as a positive control to determine the signal indicative of 100% viability after 3 days of culture. Dose-response curves and IC₅₀ values were then calculated from the data for both sets of plates (drugs arrayed in dose-response alone or the same drugs arrayed in dose-response that also included a low dose of the resistant drug used in combination). The presence of a compound that blocked the pathway mediating drug resistance of the patient cells was identified as a compound that restored sensitivity to the original drug and resulted in a significantly lower IC₅₀ value when cells were exposed to the drug combination as compared to the IC₅₀ value when either the therapeutic agent or the compound were used alone. An example of the results of the method and the dose-response curves for 256 independent drugs is illustrated in FIG. 4.

This method can identify a molecule or pathway that is responsible for mediating drug resistance in patient leukemia or ovarian cancer cells. In so doing, this approach provides a rapid means of identifying combinations of drugs that sensitize resistant cells to treatment with the compound to which the patient is resistant, which may result in enhanced cytotoxicity and efficacy when these drugs are used in combination to treat the patent in the clinic.

1. A method for identifying one or more compounds that inhibit cell growth or promote cytotoxicity of a hematological cancer or ovarian cancer from a subject comprising:
a) obtaining bone marrow or peripheral blood cells from a subject with a hematological cancer or ovarian cells from a subject with ovarian cancer;
b) pre-culturing the bone marrow or peripheral blood cells or ovarian cells for a period that allows cell death of a subset of the cells;
c) sorting the pre-cultured cells through a cell sorter to acquire a subset of viable hematological cancer or ovarian cancer cells;
d) dispensing aliquots of the viable cancer cells into individual wells of one or more multi-well tissue culture plates;
e) contacting the viable cancer cell s in each well with a unique compound selected from the compounds set forth in Table 1 or Table 2; and
f) identifying one or more compounds that inhibit cell growth or promote cytotoxicity of the contacted cells.

2. The method of claim 1, wherein the subject has leukemia and the hematological cancer cells are leukemic cells.

3. The method of claim 2, wherein the leukemia is acute myeloid leukemia.

4. The method of claim 3, wherein the subject has chemorefractory AML or newly diagnosed AML.

5. The method of claim 1, wherein the viable cells in each of at least 412 wells are contacted with a unique compound selected from the compounds set forth in Table 1.

6. The method of claim 1, wherein the viable cells in each of at least 52 wells are contacted with a unique compound selected from the compounds set forth in Table 2.

7. The method of claim 1, wherein the one or more compounds that inhibit cell growth or cytotoxicity kill at least 70% of the cells contacted with the compound.

8. The method of claim 1, wherein, after obtaining the cells from the subject, the cells are frozen and thawed prior to pre-culturing the cell s.

9. The method of claim 1, wherein the cell sorter is a fluorescence activated cell sorter (FACS).

10. The method of claim 1, wherein the multi-well tissue culture plate is a 1,536-well tissue culture plate.

11. The method of claim 10, wherein about 4,000 to about 5,000 viable cells are dispensed into each well.

12. The method of claim 1, wherein the cells are pre-cultured for at least 12 hours.

13. A method for identifying one or more compounds that sensitize refractory hematological or ovarian cancer cells from a subject comprising:
a) obtaining hematological cancer cells from bone marrow or peripheral blood cells from a subject having a hematological cancer or ovarian cancer cells from a subject with ovarian cancer, wherein the hematological or ovarian cancer cells are refractory to a therapeutic agent;
b) dispensing aliquots of the cells into individual wells of one or more multi-well tissue culture plates;
c) contacting the cells in a first subset of the wells with a combination of a low dose of the therapeutic agent to which the cells are refractory and a selected dose of a unique compound selected from the compounds set forth in Table 1 or Table 2, wherein the selected dose of each compound varies across wells in the first subset;
d) contacting the cells in a second subset of the wells with the same compounds and selected doses thereof of the first subset; and
e) identifying one or more compound s that sensitize the cells to the refractory agent by determining which compounds inhibit cell growth or promote cytotoxicity in the first subset of well of the contacted cells more than the same one or more compounds in the second subset of wells.

14. The method of claim 13, wherein the low dose of the therapeutic agent is the IC₁₀ of the therapeutic agent in inhibiting cell growth or promoting cytotoxicity of the hematological cancer cells from bone marrow or peripheral blood cells from the subject having a hematological cancer or ovarian cancer cells from the subject with ovarian cancer.

15. The method of claim 14, further comprising determining the low dose of the therapeutic agent by
f) dispensing aliquots of the cells into individual wells of one or more multi-well tissue culture plates;
g) contacting sets of wells with a first concentration, a second concentration, a third concentration, or a fourth concentration of the therapeutic agent;
h) determining which concentrations of the therapeutic agent inhibit cell growth or promote cytotoxicity to determine the IC₁₀ of the therapeutic agent.

16. The method of claim 15, wherein the dispensed cells of step f) are a subset of cells obtained in step (a).

17. The method of claim 13, wherein identifying one or more compounds that sensitize the cells to the therapeutic agent comprises determining whether the IC₅₀ of the compound is reduced in the presence of the therapeutic agent.

18. The method of claim 13, further comprising
i) pre-culturing the refractory hematological or ovarian cancer cells for a period that allows cell death of a subset of the cells;
j) sorting the pre-cultured cell s through a cell sorter to acquire a subset of viable cells for dispensing into individual wells.

19. The method of claim 13, wherein the hematologic cancer is leukemia.

20. The method of claim 19, wherein the leukemia is acute myeloid leukemia (AML).

21. The method of claim 13, wherein, after obtaining the cells from the subject, the cells are frozen and thawed prior to pre-culturing or dispensing the cells.

22. The method of claim 13, wherein the multi-well tissue culture plate is a 1,536 well tissue culture plate.

23. The method of claim 13, wherein about 4,000 to about 5,000 viable cells are dispensed into each well.

24. The method of claim 18, wherein the cells are pre-cultured for at least 12 hours.

25. A method of treating a hematological or ovarian cancer in a subject comprising:
a) identifying one or more compounds that inhibit cell growth or promote cytotoxicity of hematological cancer or ovarian cancer cells from the subject according to the method of claim 1; and
b) administering to the subject the one or more compounds that inhibit cell growth or promote cell killing of the hematological cancer cells.

26. The method of claim 25, wherein the hematological cancer is leukemia.

27. The method of claim 26, wherein the leukemia is acute myeloid leukemia (AML).

28. The method of claim 25, wherein a second anti-cancer therapy is administered to the subject.

29. The method of claim 26, wherein the anti-cancer therapy is chemotherapy, a molecularly targeted therapy, immunotherapy, radiotherapy, a pathway or selective protein-targeted inhibitor, or a bone marrow transplant

30. The method of claim 25, wherein the identified compound is selected from the group consisting of an HSP90 inhibitor, a cardiac glycoside, an HDAC inhibitor, an ion channel inhibitor, a calcium ion channel blocker, a statin, a CDK inhibitor, a proteasome inhibitor, a WNT inhibitor, a macrocyclic lactone, a lipase inhibitor, an antiparasitic, an antifungal and an antibiotic.

31.-44. (canceled)

45. The method of claim 29, wherein the molecularly targeted therapy is an inhibitor that targets AML cells with mutated FLT3.

46. The method of claim 45, wherein the mutated F133 is a FLT3-ITD or FL13-TKD mutation,

47. The method of claim 46, wherein the targeted FLT3 inhibitor is combined with a targeted inhibitor that targets JAK2 or JAK3 kinase.

48. A method of treating cancer in a subject comprising
a) administering to the subject a therapeutic agent to which the cancer is refractory and
b) administering to the subject one or more compounds that sensitize refractory hematological or ovarian cancer cells from the subject, wherein the one or more compounds is identified according to the method of claim 13.
